(19) United States
(12) Patent Application Publication
(10) Pub. No.: US 2022/0061922 A1
(43) Pub. Date: Mar. 3, 2022

(54) APPARATUS AND METHOD FOR POSTERIOR NASAL NERVE ABLATION

(71) Applicants: Acclarent, Inc., Irvine, CA (US); Biosense Webster (Israel) Ltd., Yokneam (IL)

(72) Inventors: Itzhak Fang, Irvine, CA (US); Jetmir Palushi, Irvine, CA (US); Ehsan Shameli, Irvine, CA (US); Babak Ebrahimi, Irvine, CA (US); Roozbeh Borjian, Irvine, CA (US); Behnam Amin, Mission Viejo, CA (US); Itamar Bustan, Zichron Ya'acov (IL); Helen Wolfson, Haifa (IL)

(21) Appl. No.: 17/384,855

(22) Filed: Jul. 26, 2021

Related U.S. Application Data
(60) Provisional application No. 63/069,770, filed on Aug. 25, 2020.

(51) Int. Cl. A61B 34/20; A61B 18/14

(52) U.S. Cl. CPC A61B 34/20; A61B 2018/00577; A61B 18/1492

(57) ABSTRACT

An ablation instrument includes a shaft that extends from a grip portion. A curved portion of the shaft is shaped to allow insertion into the head of a patient, and to position an ablation tip that extends from a distal tip of the shaft proximate to an ablation target, such as the posterior nasal nerve. When activated the ablation tip projects radiofrequency energy to ablate nearby tissue. An ablation assistance features of an image guided surgery navigation system is configured to segment and identify ablation targets within a set of pre-operative images. When a position tracked ablation instrument is configured for use, the system begins to monitor the proximity of the ablation tip to the ablation targets. When the tip is within an effective distance of the target for ablation, the system provides an alert and activates the instrument so that RF energy may be projected.

### PRIORITY

This application claims priority to U.S. Provisional Pat. App. No. 63/069,770, entitled “Apparatus and Method for Posterior Nasal Nerve Ablation,” filed Aug. 25, 2020, the disclosure of which is incorporated by reference herein, in its entirety.

### BACKGROUND

Rhinitis is a medical condition that presents as irritation and inflammation of the mucous membrane within the nasal cavity. The inflammation results in the generation of excessive amounts of mucus, which can cause runny nose, nasal congestion, sneezing, and/or post-nasal drip. Allergenic rhinitis is an allergic reaction to environmental factors such as airborne allergens, while non-allergenic (or “vasomotor”) rhinitis is a chronic condition that presents independently of environmental factors. Conventional treatments for rhinitis include antihistamines, topical or systemic corticosteroids, and topical anticholinergics, for example.

For cases of intractable rhinitis in which the symptoms are severe and persistent, an additional treatment option is the surgical removal of a portion of the vidian (or “pterygoid”) nerve—a procedure known as vidian neurectomy. The theoretical basis for vidian neurectomy is that rhinitis is caused by an imbalance between parasympathetic and sympathetic innervation of the nasal cavity, and the resultant over stimulation of mucous glands of the mucous membrane. Vidian neurectomy aims to disrupt this imbalance and reduce nasal mucus secretions via surgical treatment of the vidian nerve. However, in some instances, vidian neurectomy can cause collateral damage to the lacrimal gland, which is innervated by the vidian nerve. Such damage to the lacrimal gland may result in long-term health complications for the patient, such as chronic dry eye. Posterior nasal neurectomy, or surgical removal of a portion of the posterior nasal nerves, may be an effective alternative to vidian neurectomy for treating intractable rhinitis.

FIG. 1 depicts a left sagittal view of a portion of a patient's head, showing the nasal cavity (10), the frontal sinus (12), the sphenoid sinus (14), and the sphenoid bone (16). The nasal cavity (10) is bounded laterally by the nasal wall (18), which includes an inferior turbinate (20), a middle turbinate (22), and a superior turbinate (24). The vidian nerve (32) resides within the vidian (or “pterygoid”) canal (30), which is defined in part by the sphenoid bone (16) and is located posterior to the sphenoid sinus (14), approximately in alignment with the middle turbinate (22). The Sphenopalatine foramen (39) is located proximate to the posterior end of the middle turbinate (22) connection to the lateral wall. The vidian nerve (32) is formed at its posterior end by the junction of the greater petrosal nerve (34) and the deep petrosal nerve (36); and joins at its anterior end with the pterygopalatine ganglion (38), which is responsible for regulating blood flow to the nasal mucosa. The posterior nasal nerves (40) join with the pterygopalatine ganglion (38) and extend through the region surrounding the inferior turbinate (20).

While instruments and methods for performing vidian neurectomies and posterior nasal neurectomies are known, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a left sagittal view of a portion of a patient's head, showing details of certain paranasal sinuses and nerves, including the vidian nerve and the posterior nasal nerve;

FIG. 2 depicts a schematic diagram of an image guided surgery system configured to assist with ablation;

FIG. 3A depicts a perspective view of an ablation instrument usable with the system of FIG. 2;

FIG. 3B depicts a side elevation view of the ablation instrument of FIG. 3A;

FIG. 4A depicts a perspective view of an alternate ablation instrument that includes a shapeable shaft and that usable is with the system of FIG. 2;

FIG. 4B depicts a side elevation view of the ablation instrument of FIG. 4A;

FIG. 5 is a flowchart of a set of steps that could be performed to provide navigation assistance during an ablation procedure;

FIG. 6 shows an exemplary interface that may be displayed during some of the steps of FIG. 5;

FIG. 7A shows an exemplary interface that may be displayed during some of the steps of FIG. 5 to indicate a tracked ablation instrument;

FIG. 7B shows an exemplary interface that may be displayed during some of the steps of FIG. 5 to indicate navigation of a tracked ablation instrument;

FIG. 7C shows an exemplary interface that may be displayed during some of the steps of FIG. 5 to indicate continued navigation of a tracked ablation instrument;

FIG. 7D shows an exemplary interface that may be displayed during some of the steps of FIG. 5 to indicate arrival of a tracked ablation instrument at a target;

FIG. 7E shows an exemplary interface that may be displayed during some of the steps of FIG. 5 with an overlay of an ablation instrument;

FIG. 8 shows another exemplary interface that may be displayed during some of the steps of FIG. 5 to indicate arrival of a tracked ablation instrument at a target; and

FIG. 9 shows an exemplary interface that may be displayed during some of the steps of FIG. 5 to indicate completion of an ablation procedure.

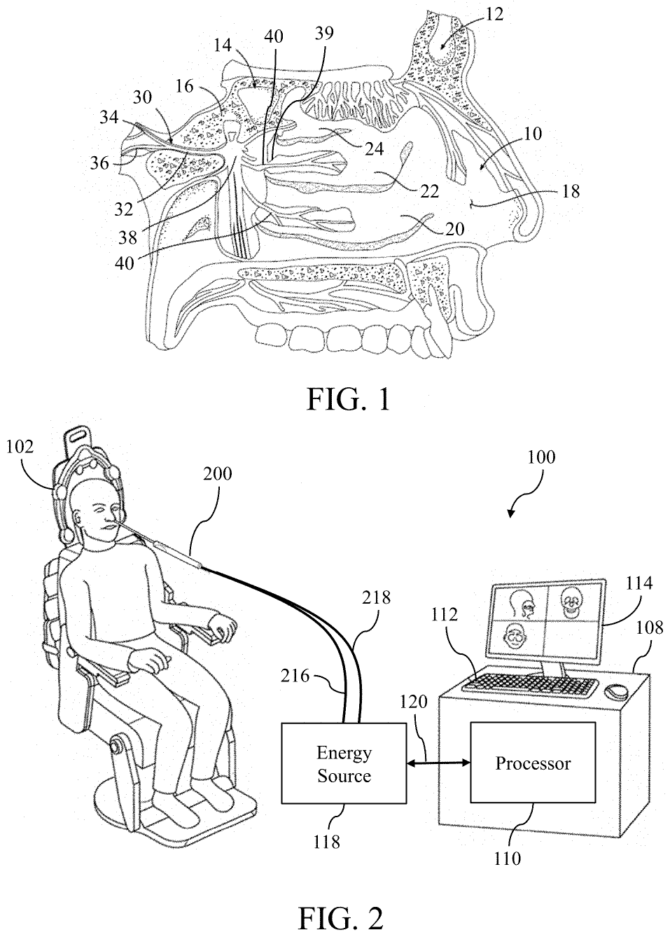

### DETAILED DESCRIPTION

The inventors have conceived of novel technology that, for the purpose of illustration, is disclosed herein as applied in the context of devices and methods for surgical treatment. While the disclosed applications of the inventors' technology satisfy a long-felt but unmet need in the art of devices and methods for surgical treatment, it should be understood that the inventors' technology is not limited to being implemented in the precise manners set forth herein, but could be implemented in other manners without undue experimentation by those of ordinary skill in the art in light of this disclosure. Accordingly, the examples set forth herein should be understood as being illustrative only, and should not be treated as limiting.

I. Exemplary System for Posterior Nasal Nerve Ablation

When performing a medical procedure, it may be desirable to have information regarding the position of an instrument within the patient, particularly when the instrument is in a location where it is difficult or impossible to obtain an endoscopic view of a working element of the instrument. FIG. 2 shows an exemplary IGS navigation system (100) enabling an ENT procedure, such as a posterior nasal nerve ablation, to be performed using image guidance. In addition to or in lieu of having the components and operability described herein IGS navigation system (100) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein.

IGS navigation system (100) of the present example comprises a field generator assembly (102) operable to generate alternating magnetic fields of different frequencies around the patient to produce a tracked area that the IGS navigation system (100) associates a coordinate system with. In this example, an ablation instrument (200) is inserted into the head of the patient. Examples of features and operability of instrument (200) will be described in greater detail below. In some implementations, the patient may be seated on a chair, which may be configured and operable in accordance with at least some of the teachings of U.S. Pat. No. 10,561,370, entitled “Apparatus to Secure Field Generating Device to Chair,” issued Feb. 18, 2020, the disclosure of which is incorporated by reference herein.

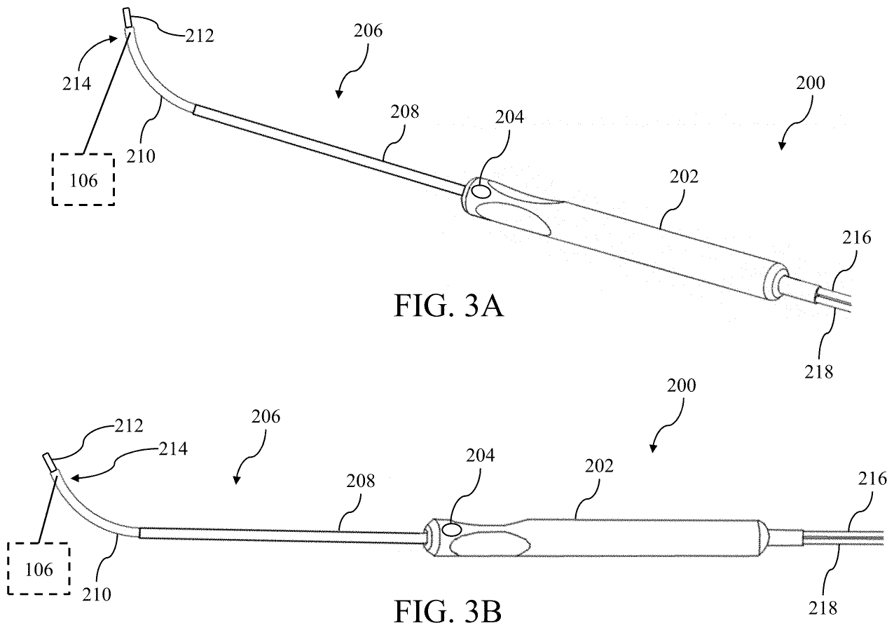

IGS navigation system (100) of the present example further comprises a processor (110), which controls the field generator assembly (102) and other elements of IGS navigation system (100). For instance, processor (110) is operable to drive field generators of the field generator assembly (102) to generate alternating electromagnetic fields; and process signals received from a sensor of ablation instrument (200) (e.g., sometimes positioned in a distal tip of ablation instrument (200) that is advanced into the patient, as illustrated by a position sensor (106) shown in FIGS. 3A and 3B) to determine the location of the sensor within the head of the patient. Processor (110) comprises one or more processing units (e.g., a set of electronic circuits arranged to evaluate and execute software instructions using combinational logic circuitry or other similar circuitry) communicating with one or more memories. Processor (110) of the present example is mounted in a console (108), which comprises operating controls (112) that include a keypad and/or a pointing device such as a mouse or trackball. A physician uses operating controls (112) to interact with processor (110) while performing the surgical procedure.

The position sensor (106) of ablation instrument (200) is responsive to positioning within the alternating magnetic fields generated by field generators in order to generate data usable to determine the position of the sensor within the magnetic fields. By way of example only, position sensor (106) may include one or more coils. Signals produced by the position sensor (106) may be communicated to the processor (110) wirelessly, or by an electrical connection such as a tracking cable (216) that couples ablation instrument (200) to the processor (110) (e.g., either directly or indirectly through another device such as an energy source (118) that provides power for ablation, and which itself is in communication with the processor (110) via a data connection (120)).

Processor (110) uses software stored in a memory of processor (110) to calibrate and operate IGS navigation system (100). Such operation includes driving field generators, processing data from the position sensor (106), processing data from operating controls (112), and driving display screen (114). Processor (110) is further operable to provide video in real time via display screen (114), showing the position of the distal end of ablation instrument (200) in relation to a video camera image of the patient's head, a CT scan image of the patient's head, and/or a computer generated three-dimensional model of the anatomy within and adjacent to the patient's nasal cavity. Display screen (114) may display such images simultaneously and/or superimposed on each other during the surgical procedure. Such displayed images may also include graphical representations of instruments that are inserted in the patient's head, such as ablation instrument (200), such that the operator may view the virtual rendering of the instrument at its actual location in real time. By way of example only, display screen (114) may provide images in accordance with at least some of the teachings of U.S. Pat. No. 10,463,242, entitled “Guidewire Navigation for Sinuplasty,” issued Nov. 5, 2019, the disclosure of which is incorporated by reference herein. In the event that the operator is also using an endoscope, the endoscopic image may also be provided on display screen (114).

Ablation instrument (200) may be usable during a surgical procedure to ablate or otherwise affect tissue within the nasal cavity (10), such as the posterior nasal nerve (40). Examples of ablation techniques include radiofrequency (RF) ablation, wherein energy is delivered to the target via radio frequency, and cryoablation, wherein a freezing substance is delivered to the target. Where ablation instrument (200) is configured for RF ablation of tissue, it may be coupled to the energy source (118) which may be, for example, an RF generator or other RF source. RF energy produced by the energy source (118) is transmitted to ablation instrument (200) via an energy cable (218) and emitted at a distal tip of ablation instrument (200) in order to ablate nearby tissue. The energy source (118) is in communication with the processor (110) via the data connection (120), which may be, for example, a wireless or wired data connection (e.g., Wi-Fi, Ethernet). Information exchanged via the data connection (120) may include, for example, coordinate system and location tracking information for ablation instrument (200) and other tracked objects, signals to enable, disable, or otherwise reconfigure the operation of the energy source (118) and/or ablation instrument (200), and information relating to use of the surgical instrument (e.g., parameters describing power output, instances and duration of activations, etc.).

II. Exemplary Ablation Instrument

FIGS. 3A and 3B each show examples of an ablation instrument (200) that may be used with system (100). The ablation instrument (200) includes a probe grip (202) that may have a shape, surface materials, and surface features that aid in gripping and directing the instrument (200). In some implementations, the probe grip (202) may include a button (204) that may be actuated by a user to activate and/or deactivate output of RF energy from the ablation instrument (200). In some other versions, a footswitch (not shown) is used to provide selective activation of RF energy. A shaft (206) extends from the probe grip (202) and includes a linear portion (208) and a curved portion (210). An ablation tip (212) emerges from the curved portion (210) of the shaft and is configured to apply RF energy to tissue when the energy source (118) and/or ablation instrument (200) are activated. In some versions, ablation tip (212) includes a single electrode and is configured to cooperate with a ground pad that is in contact with the patient's skin to apply monopolar RF energy to tissue. In some other versions, ablation tip (212) includes two or more electrodes that are operable to apply bipolar RF energy to tissue.

The position sensor (106) may be embedded within or coupled to the shaft (206), and may be located, for example, at a distal end (214) of the shaft (206) proximate to the ablation tip (212). As has been described, the position sensor (106) may interact with a magnetic field or other characteristic or signal present within the tracked area to produce signals indicating the position of the distal end (214) and, resultingly, the ablation tip (212) and other portions of the ablation instrument (200), within the magnetic field.

A tracking cable (216) extends from the probe grip (202) and may be coupled to (e.g., wirelessly or via a cable) the energy source (118), the processor (110), or both, and may be configured to exchange electrical signals and data with a coupled device. Data transmitted via the tracking cable (216) may include, for example, data or signals produced by the position sensor (106), control signals configured to cause the energy source (118) to activate and/or deactivate and control the transmission of RF energy from the ablation tip (212), and usage parameters related to activation and use of the surgical features of the ablation instrument (200).

An energy cable (218) also extends from the probe grip (202) and may be coupled to the energy source (118) and configured to transmit received RF energy to the ablation tip (212). The energy cable (218) may be coupled with a proximal end of the shaft (206) or another cable that itself is coupled to the proximal end of the shaft (206), such that received RF energy travels along a channel within the probe grip (202). The probe grip (202) and the linear (208) and curved (210) portions of the shaft (206) may each include one or more isolating layers that are configured to prevent or mitigate undesired transmission of the RF energy, such that all or substantially all of the received RF energy is directed to the ablation tip (212) rather than being projected from the shaft (206) or the probe grip (202). In some implementations the isolating layer may include one or more non-conductive or shielding materials, and the materials of the shaft (206) and probe grip (202) may themselves be non-conductive, save for the embedded channel that connects the ablation tip (212) to the energy cable (218).

One or more characteristics of the shaft (206) may be selected to aid in insertion into the head of a patient, positioning of the ablation tip (212) at the posterior nasal nerve (40) or other surgical site, or both. For example, the length of the linear portion (208) and curved portion (210) may each be selected to allow the shaft (206) to be inserted to the depth needed for the ablation procedure, and the curvature of the curved portion (210) may be selected based upon the surrounding anatomy that is traversed during navigation to the posterior nasal nerve (40) so that no artificial channels or passages need to be created during the ablation procedure. These one or more characteristics of the shaft (206) may be selected based upon average anatomical characteristics across all patients; or may be customized such that the shaft (206) may be produced with particular characteristics for each patient and/or procedure. Shafts (206) having such customized characteristics may be produced as linear, and may be later malleably formed into the desired curvature, or may be produced using additive manufacturing techniques (e.g., to produce a non-conductive housing that includes a channel into which an RF transmitting circuit may be inserted), or may be produced in other ways.

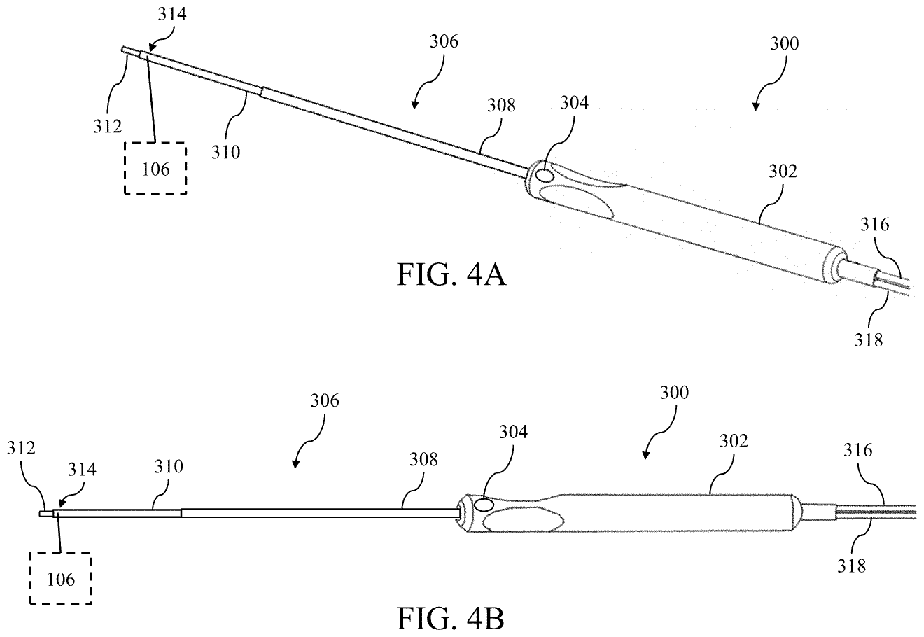

FIGS. 4A and 4B each show examples of an alternate ablation instrument (300) having similar components and features as the ablation instrument (200), including a probe grip (302), a button (304), a shaft (306), a position sensor (106) at a distal end (314) of the shaft (306), an ablation tip (312), a tracking cable (316), and an energy cable (318), each previously described in the context of FIGS. 3A and 3B. The ablation instrument (300) does not include a preformed bend in the shaft (306), and instead includes a linear portion (308) and an adjustable portion (310) from which the ablation tip (312) emerges. As with the previously described linear portion (208) and the curved portion (210), the portions of the shaft (306) may include isolating layers, non-conductive materials, and shielding layers to ensure that all or substantially all of the received RF energy is directed to the ablation tip (312).

The adjustable portion (310) of the shaft is configured to be shaped to a desired curvature to aid in positioning the ablation tip (312) at the target site (e.g., the posterior nasal nerve (40)) when the shaft (306) is inserted as part of an ablation procedure. Adjustment of the adjustable portion (310) may be accomplished in varying ways. In some implementations, the adjustable portion (310) may include malleable materials that may be formed (e.g., either at room temperature, or with heat treatment) using a hand or tool to produce the desired shape. In some implementations, the adjustable portion (310) may include flexible materials or articulated segmentation that allows for varying shapes and curvature to be produced via active steering using control wires (e.g., a wire running along the shaft and coupled to a position near the distal end such that retraction of the wire flexes the shaft portion) or electroactive materials (e.g., polymers or metals that change in shape in response to electrical inputs). Other devices and techniques for adjusting the adjustable portion (310) exist and will be apparent to those skilled in the art in light of this disclosure.

III. Exemplary Method for Navigation Assisted Ablation

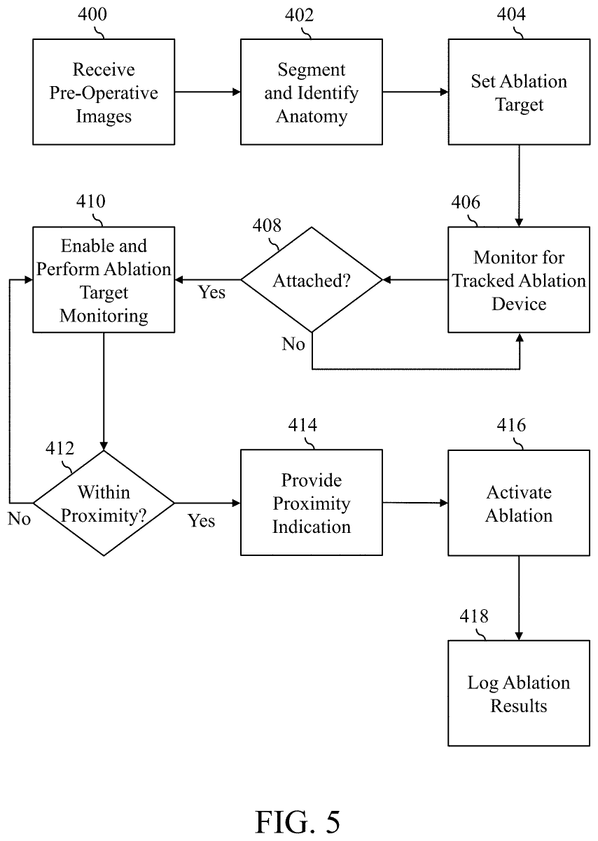

FIG. 5 shows a flowchart of a set of steps that could be performed to provide navigation assistance during an ablation procedure, and that may be performed with the system (100) and ablation instruments (200, 300) described above. A set of pre-operative images may be received (400) that includes image slices (e.g., CT images), three dimensional models, or other computed images of the patient anatomy. These images may be registered to the coordinate system of the IGS navigation system (100) and correlated with the patient and any tracked instruments or devices.

The images may also be segmented (402) to identify, within image slices and/or three-dimensional models, one or more patient anatomical structures that are associated with the surgical procedure. This may include, for example, identifying the posterior nasal nerve (40) and/or other patient anatomy proximate to the posterior nasal nerve (40) which an ablation tip (e.g., the ablation tip (212, 312)) may be navigated to in order to transmit RF energy to the posterior nasal nerve (40). As an example, this may include identifying the Sphenopalatine foramen, or identifying a location at the posterior end of the middle turbinate (22) connection to the lateral wall, as close as possible to the Sphenopalatine foramen instead of or in addition to identifying the posterior nasal nerve (40). Segmentation of the pre-operative images may be performed using a segmentation algorithm. In some implementations, the segmentation algorithm may utilize a machine learning process configured to be trained over time using image sets and physician feedback (e.g., manual segmentation of patient anatomy, confirmation of automatically segmented and identified anatomy) received from a plurality of procedure sites using the system (100).

An ablation target may be set (404) on one or more of the segmented (402) and identified anatomy. This may include setting (404) a target on the posterior nasal nerve (40), the Sphenopalatine foramen (39), or a location near one or both that may be suitable for transmitting RF energy to the posterior nasal nerve (40). The size and shape of the set (404) target may be varied based upon the capabilities of the ablation instrument (200,300) and the characteristics of the surrounding patient anatomy. For example, an ablation target may be a single coordinate within the coordinate system; or may be a two-dimensional or three-dimensional shape having various size, shape, and symmetry. In one example, the ablation target may be a spherical zone that includes a plurality of points of the patient anatomy from which RF energy transmitted by an ablation tip will be received at the posterior nasal nerve (40). The ablation target may be set (404) based upon a manual selection of one or more anatomical structures by a user (e.g., a selection of segmented and identified anatomy from a list), or may be set (404) automatically based upon configurations or records describing the type of procedure that is to be performed (e.g., the system (100) may receive input, from a user or another system, indicating that a posterior nasal nerve ablation procedure is being performed, and may automatically set (404) an ablation target on the posterior nasal nerve (40) in response).

The system (100) may then monitor (406) for the presence, configuration, or use of a tracked ablation device during a procedure associated with the set (404) ablation target. Use of a tracked ablation device may be determined by a manual input from a user (e.g., interacting with the operating controls (112) or the button (204)), or may be determined automatically based upon information received from the ablation instrument (200, 300), the energy source (118), or both. In some implementations, the ablation instrument (200, 300) may include a memory (e.g., an EEPROM or other ROM/RAM) within the probe grip (202) and communicatively coupled to the tracking cable (216). Information stored on the memory may uniquely identify (e.g., a serial number) the ablation instrument (200, 300), and may also describe or indicate a type and capability of the instrument (e.g., a model number). This identifying information may be provided to the processor (110) and/or energy source (118) via the tracking cable (216) or other data connection, and may be used to determine that the associated device is a tracked ablation device (e.g., via a search of local or remote database, or analysis of characteristics of a serial number and/or model number).

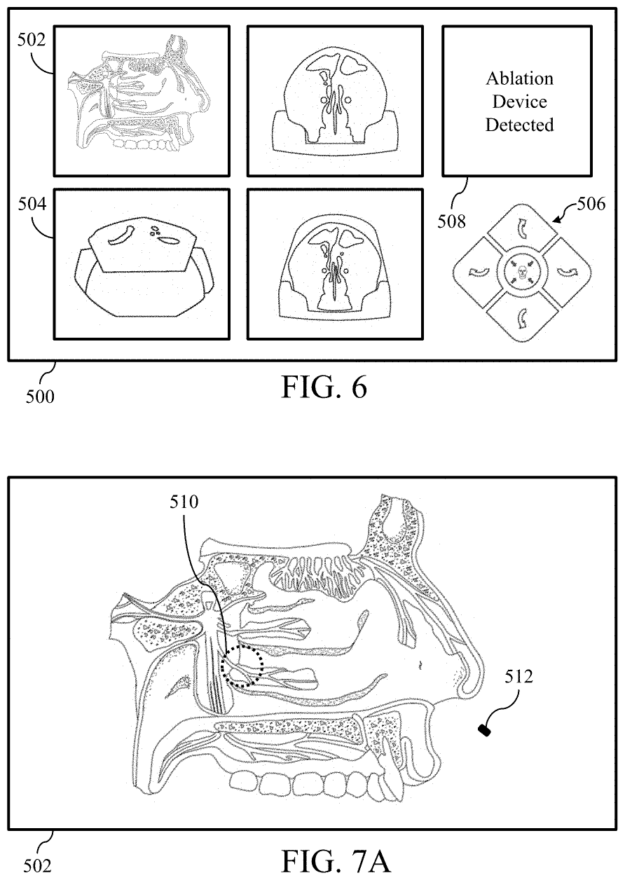

When a tracked ablation device is attached (408) configured for use with the system (100), the IGS navigation software may enable (410) and begin to perform ablation target monitoring for that device. FIG. 6 shows an example of an interface (500) that may be provided by the IGS navigation software via the display screen (114) or another display during ablation target monitoring. An image view (502) may display a pre-operative image slice or model from a first perspective, while other image views (504) may show image slices or models from differing perspectives. The image views (502) may be overlaid with information and icons indicating the location of tracked instruments relative to the shown patient anatomy. A set of controls (506) are usable to navigate image slices and models. A status pane (508) is configured to display status information for the system (100), and may be configured as a static portion of the interface, or a dynamic element (e.g., a pop-up window or other dynamic graphic that appears to alert a user to a change in status).

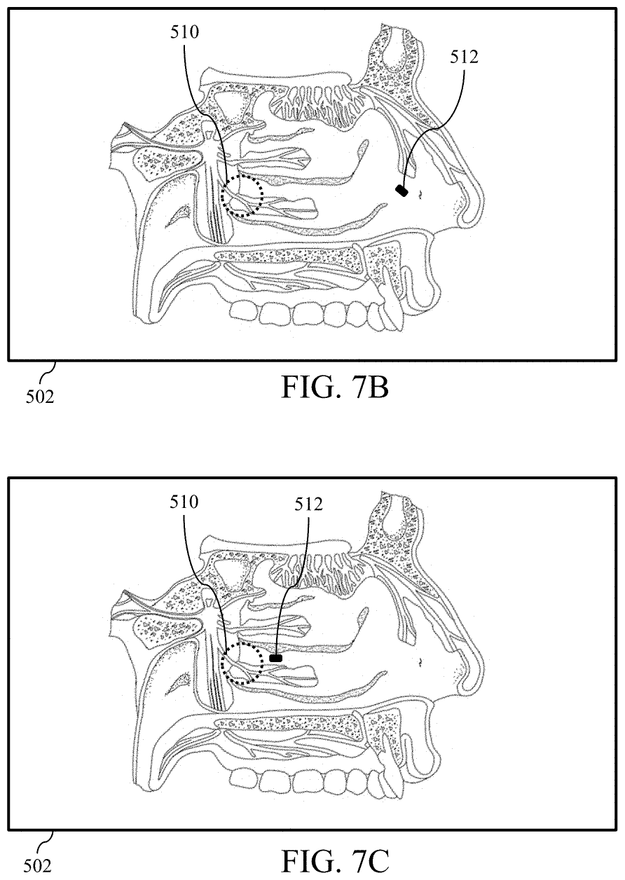

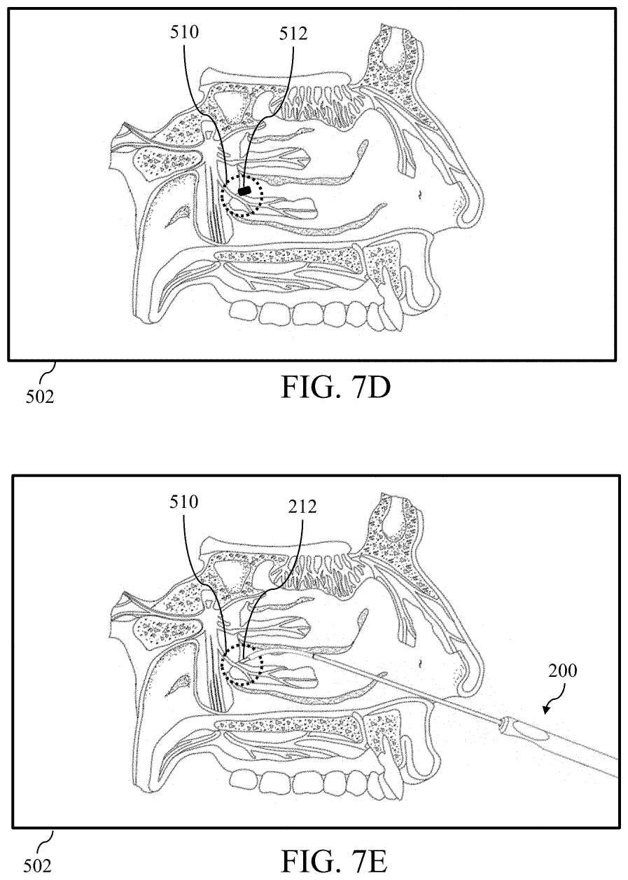

As can be seen in FIG. 6, the status pane (508) is displaying a message indicating that a device has been detected and that ablation target monitoring has been enabled (410). Referring back to FIG. 5, while enabled (410), the system (100) may regularly determine the proximity of the tracked ablation device to the ablation target, and determine whether the ablation tip (212, 312) is within a configured proximity (412) of the ablation target that will allow for RF energy to be effectively transmitted to the target anatomy (e.g., the posterior nasal nerve (40)). This may include regularly determining the position of the ablation tip (212, 312) based upon feedback from one or more position sensors (106), as has been described, and overlaying an icon on one or more of the image views (502, 504) to show the current position. FIG. 7A shows an example of the image view (502), such as may be displayed via the interface (500). An ablation target zone (510) has been overlaid upon the area that the ablation instrument must be navigated to in order to perform the procedure, and an instrument indicator (512) has been overlaid to indicate the instrument's current position. FIGS. 7B and 7C show additional examples of the image view (502), each with the position of the instrument indicator (512) updated in real time as ablation tip (212, 312) is navigated towards the ablation target zone (510).

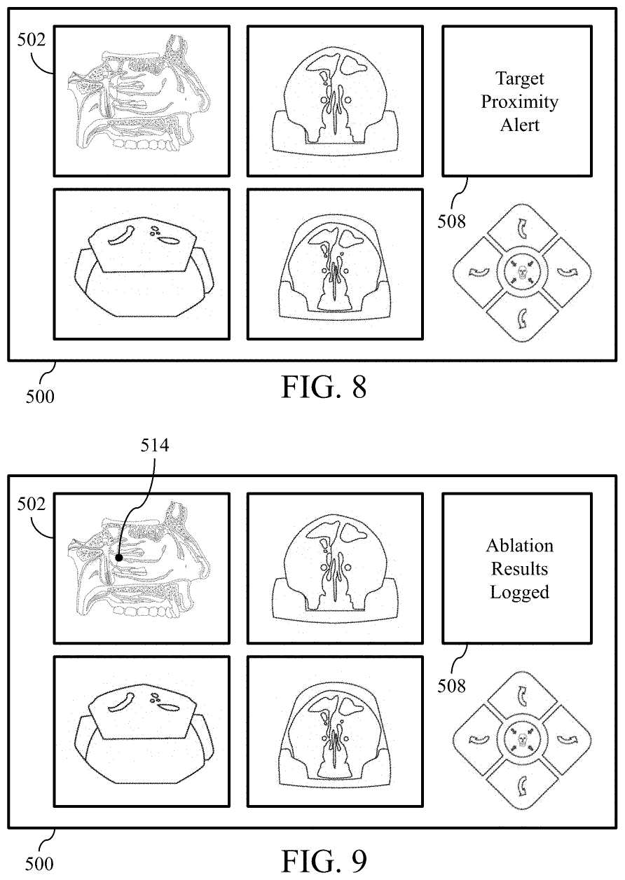

When the ablation tip (212, 312) is determined to be within the proximity (412) of the target, as illustrated in FIG. 7D, the system may provide (414) a proximity indication, as shown in FIG. 5. The proximity indication may be a visual, audible, haptic, or other form of feedback to alert and indicate to a user of the ablation instrument (200, 300) that the ablation tip (212, 312) has reached a position at which the ablation procedure may be performed. FIG. 7E illustrates an approximate position of the ablation instrument (200) relative to the patient anatomy and ablation target zone (510). In some implementations, the image view (502) may display an overlay of the virtual ablation instrument (200) as shown in FIG. 7E, instead of or in addition to indicators such as the graphic indicator (512). In some implementations, the provided (414) indication may be an alert, notification, or change in status of the interface (500), as illustrated in FIG. 8, in which the status pane (508) now indicates a target proximity alert.

As shown in FIG. 5, the ablation feature of the ablation instrument (200, 300) may then be activated (416), causing RF energy to be applied by the ablation tip (212, 312) to the ablation target. Activation (416) of the ablation feature may be based upon manual input by a user, automated input from the processor (110) or energy source (118), or both. As an example, in some implementations the RF feature of the ablation instrument (200, 300) may be entirely inoperable as a safety feature, and may only become usable when the ablation tip (212, 312) is determined to be within proximity (412) of the target zone. In some implementations, the ablation feature may become operable and activate automatically based upon the tracked proximity (412). In some implementations, the ablation feature may become operable automatically based upon the tracked proximity (412), and then may be manually activated by a user via an interaction with the energy source (118) or interaction with the button (204, 304).

After activation (416) and performance of the ablation procedure (e.g., a single ablation or multiple ablations) results of the ablation may be logged (418) and saved. This may include recording parameters such as the ablation instrument's tracked position at the time of use, the duration of use, the RF energy output level during the use, identifying information for the ablation instrument (200, 300) in use, and other information. Logged (418) information may be received from the ablation instrument (200, 300), where it may be stored on a memory or produced by a processor, or may be received from the energy source (118), where it may be stored on a memory or produced by a processor based on the energy drawn, produced, and transmitted to the ablation instrument (200, 300) during use. In addition to storing such information in logs, it may also be used to update the interface (500).

FIG. 9 shows an example of the interface (500) after results of an ablation are logged (418). An ablation indicator (514), illustrated as a filled circle, is positioned on the image view (502) at the location where the ablation feature (e.g., projection of RF energy from the ablation tip (212)) was used. The ablation indicator (514) may have a varying shape, size, color, or other characteristic based upon parameters of the use. For example, a green circle may indicate that prior RF energy projection was below a safe threshold and further ablation is possible, while a red circle may indicate that the threshold has been exceeded and further ablation at that location should not be performed. Prior ablation use may also be indicated and described with text associated with the indicator (514), which may appear in a popup window or other screen; or may be displayed via the status pane (508).

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An image guided surgery system comprising: (a) an ablation instrument comprising: (i) a body, (ii) a shaft extending from the body, (iii) an ablation tip at a distal end of the shaft, the ablation tip being operable to apply radiofrequency (RF) energy to tissue; (b) a position sensor coupled to the shaft and configured to produce signals based on a position of the position sensor within a tracked area; (c) a processor configured to: (i) identify an ablation target within a set of pre-operative images associated with a patient, (ii) determine a current position of the ablation tip within the tracked area based on a set of position signals from the position sensor, and (iii) while an ablation targeting monitoring mode is active, determine that the current position of the ablation tip is at the ablation target and, in response, provide a proximity indication to a user of the ablation instrument.

### EXAMPLE 2

The system of Example 1, wherein the shaft comprises a curved portion at the distal end, and wherein the shape of the curved portion is configured to position the ablation tip at the ablation target when the shaft is inserted into the head of the patient.

### EXAMPLE 3

The system of Example 2, wherein the shape of the curved portion is rigid.

### EXAMPLE 4

The system of any one or more of Examples 2 through 3, wherein curved portion comprises a flexibility feature that allows the shape of the curved portion to be determined by the user.

### EXAMPLE 5

The system of any one or more of Examples 1 through 4, the ablation instrument further comprising a button on the body that is configured to selectively control the transmission of RF energy from the ablation tip.

### EXAMPLE 6

The system of Example 5, wherein the button is configured to selectively transmit a signal to an energy source that is configured to cause the energy source to transmit RF energy to the ablation tip.

### EXAMPLE 7

The system of any one or more of Examples 1 through 6, the ablation instrument further comprising an isolation layer positioned around the shaft, wherein the isolation layer is configured to mitigate the transmission of RF energy from the shaft into the surrounding environment.

### EXAMPLE 8

The system of any one or more of Examples 1 through 7, wherein the processor is further configured to, when identifying the ablation target: (i) segment the set of pre-operative images using a segmentation algorithm to identify positions of a set of anatomical structures, and (ii) select the ablation target based upon the set of anatomical structures and data describing a procedure being performed on the patient.

### EXAMPLE 9

The system of Example 8, wherein the segmentation algorithm comprises a machine-learning process that is configured to segment and identify anatomical structures based upon a training dataset, wherein the training dataset is produced from a plurality of pre-operative images and a plurality of historical segmentation results.

### EXAMPLE 10

The system of Example 9, wherein the processor is further configured to select the ablation target based upon a posterior nasal nerve identified within the set of anatomical structures.

### EXAMPLE 11

The system of any one or more of Examples 9 through 10, wherein the processor is further configured to select the ablation target based upon: (i) a middle turbinate connection identified within the set of anatomical structures, and (ii) a Sphenopalatine foramen identified within the set of anatomical structures.

### EXAMPLE 12

The system of any one or more of Examples 1 through 11, wherein the processor is further configured to: (i) display an interface on a display, the interface comprising an image that is based on the set of pre-operative images, and (ii) overlay an instrument indicator on the image based on the current position.

### EXAMPLE 13

The system of Example 12, wherein the processor is further configured to: (i) when RF energy is transmitted from the ablation tip, store a set of parameters that describe a location and a transmission of RF energy from the ablation tip, and (ii) overlay an ablation indicator on the image based on the set of parameters.

### EXAMPLE 14

The system of any one or more of Examples 1 through 13, wherein the proximity indication comprises one or more of: (i) an audible alert from a sound device, (ii) a visual alert from a display, or (iii) a haptic alert from the ablation instrument.

### EXAMPLE 15

The system of any one or more of Examples 1 through 14, wherein the processor is further configured to detect when the ablation instrument is in use and, in response, activate the ablation target monitoring mode.

### EXAMPLE 16

A method comprising: (a) selecting an ablation instrument that includes a shaft with a curved portion, and an ablation tip at a distal end of the curved portion; (b) configuring an image guided surgery system to track a position sensor that is coupled to the shaft and configured to produce signals based on its position within a tracked area; (c) with a processor, identifying an ablation target within a set of pre-operative images associated with a patient; (d) determining a current position of the ablation tip within the tracked area based on a set of position signals from the position sensor; and (e) determining the current position of the ablation tip is at the ablation target and, in response, providing a proximity indication to a user of the ablation instrument.

### EXAMPLE 17

The method of Example 16, further comprising selecting the ablation instrument based on the curved portion having a shape that positions the ablation tip at the ablation target when the shaft is inserted into the head of the patient.

### EXAMPLE 18

The method of any one or more of Examples 16 through 17, further comprising identifying the ablation target based upon an identified position of the posterior nasal nerve within the set of pre-operative images.

### EXAMPLE 19

The method of any one or more of Examples 16 through 18, further comprising identifying the ablation target based upon identified positions of a middle turbinate connection and a Sphenopalatine foramen within the set of pre-operative images.

### EXAMPLE 20

An image guided surgery system comprising one or more processors configured to: (i) identify an ablation target within a set of pre-operative images associated with a patient, (ii) detect when an ablation instrument is in use and, in response, enable an ablation target monitoring mode, (iii) determine a current position of an ablation tip of the ablation instrument within the tracked area based on a set of position signals received from a position sensor of the ablation instrument, (iv) determine that the current position of the ablation tip is at the ablation target and, in response, provide a proximity indication to a user of the ablation instrument, and (v) after providing the proximity indication, enable the transmission of radiofrequency (RF) energy from an energy source to the ablation tip.

V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An image guided surgery system comprising:
(a) an ablation instrument comprising:
(i) a body,
(ii) a shaft extending from the body, and
(iii) an ablation tip at a distal end of the shaft, the ablation tip being operable to apply radiofrequency (RF) energy to tissue;
(b) a position sensor coupled to the shaft and configured to produce signals based on a position of the position sensor within a tracked area; and
(c) a processor configured to:
(i) identify an ablation target within a set of pre-operative images associated with a patient,
(ii) determine a current position of the ablation tip within the tracked area based on a set of position signals from the position sensor, and
(iii) while an ablation targeting monitoring mode is active, determine that the current position of the ablation tip is at the ablation target and, in response, provide a proximity indication to a user of the ablation instrument.

2. The system of claim 1, wherein the shaft comprises a curved portion at the distal end, and wherein the shape of the curved portion is configured to position the ablation tip at the ablation target when the shaft is inserted into the head of the patient.

3. The system of claim 2, wherein the shape of the curved portion is rigid.

4. The system of claim 2, wherein curved portion comprises a flexibility feature that allows the shape of the curved portion to be determined by the user.

5. The system of claim 1, the ablation instrument further comprising a button on the body that is configured to selectively control the transmission of RF energy from the ablation tip.

6. The system of claim 5, wherein the button is configured to selectively transmit a signal to an energy source that is configured to cause the energy source to transmit RF energy to the ablation tip.

7. The system of claim 1, the ablation instrument further comprising an isolation layer positioned around the shaft, wherein the isolation layer is configured to mitigate the transmission of RF energy from the shaft into the surrounding environment.

8. The system of claim 1, wherein the processor is further configured to, when identifying the ablation target:
(i) segment the set of pre-operative images using a segmentation algorithm to identify positions of a set of anatomical structures, and
(ii) select the ablation target based upon the set of anatomical structures and data describing a procedure being performed on the patient.

9. The system of claim 8, wherein the segmentation algorithm comprises a machine-learning process that is configured to segment and identify anatomical structures based upon a training dataset, wherein the training dataset is produced from a plurality of pre-operative images and a plurality of historical segmentation results.

10. The system of claim 9, wherein the processor is further configured to select the ablation target based upon a posterior nasal nerve identified within the set of anatomical structures.

11. The system of claim 9, wherein the processor is further configured to select the ablation target based upon:
(i) a middle turbinate connection identified within the set of anatomical structures, and
(ii) a Sphenopalatine foramen identified within the set of anatomical structures.

12. The system of claim 1, wherein the processor is further configured to:
(i) display an interface on a display, the interface comprising an image that is based on the set of pre-operative images, and
(ii) overlay an instrument indicator on the image based on the current position.

13. The system of claim 12, wherein the processor is further configured to:
(i) when RF energy is transmitted from the ablation tip, store a set of parameters that describe a location and a transmission of RF energy from the ablation tip, and
(ii) overlay an ablation indicator on the image based on the set of parameters.

14. The system of claim 1, wherein the proximity indication comprises one or more of:
(i) an audible alert from a sound device,
(ii) a visual alert from a display, or
(iii) a haptic alert from the ablation instrument.

15. The system of claim 1, wherein the processor is further configured to detect when the ablation instrument is in use and, in response, activate the ablation target monitoring mode.

16. A method comprising:
(a) selecting an ablation instrument that includes a shaft with a curved portion, and an ablation tip at a distal end of the curved portion;
(b) configuring an image guided surgery system to track a position sensor that is coupled to the shaft and configured to produce signals based on its position within a tracked area;
(c) with a processor, identifying an ablation target within a set of pre-operative images associated with a patient;
(d) determining a current position of the ablation tip within the tracked area based on a set of position signals from the position sensor; and
(e) determining the current position of the ablation tip is at the ablation target and, in response, providing a proximity indication to a user of the ablation instrument.

17. The method of claim 16, further comprising selecting the ablation instrument based on the curved portion having a shape that positions the ablation tip at the ablation target when the shaft is inserted into the head of the patient.

18. The method of claim 16, further comprising identifying the ablation target based upon an identified position of the posterior nasal nerve within the set of pre-operative images.

19. The method of claim 16, further comprising identifying the ablation target based upon identified positions of a middle turbinate connection and a Sphenopalatine foramen within the set of pre-operative images.

20. An image guided surgery system comprising one or more processors configured to:
(i) identify an ablation target within a set of pre-operative images associated with a patient,
(ii) detect when an ablation instrument is in use and, in response, enable an ablation target monitoring mode,
(iii) determine a current position of an ablation tip of the ablation instrument within the tracked area based on a set of position signals received from a position sensor of the ablation instrument,
(iv) determine that the current position of the ablation tip is at the ablation target and, in response, provide a proximity indication to a user of the ablation instrument, and
(v) after providing the proximity indication, enable the transmission of radiofrequency (RF) energy from an energy source to the ablation tip.
